(19) United States
(12) Patent Application Publication
(10) Pub. No.: US 2012/0214834 A1
(43) Pub. Date: Aug. 23, 2012

(54) IMMUNE ENHANCEMENT COMPOSITION AND KIT AND USE THEREOF

(76) Inventor: Pei-Ru LIAO, Taipei City (TW)

(21) Appl. No.: 13/238,503

(22) Filed: Sep. 21, 2011

(30) Foreign Application Priority Data
Feb. 23, 2011 (TW) 100106087

(51) Int. Cl. A61K 31/4706; A61P 37/04; A61P 31/12; A61P 35/00; A61P 31/00

(52) U.S. Cl. 514/291

(57) ABSTRACT

An immune enhancement composition is disclosed. The immune enhancement composition includes rapamycin, substituted quinoline and a pharmaceutically acceptable carrier, diluent, excipient or a combination thereof. An immune enhancement kit containing the two aforementioned compounds and a use of a combination containing the two aforementioned compounds as an immune enhancement composition are also disclosed. The application of the composition, kit and use is advantageous for promoting the activity of the immune system.

### CROSS REFERENCE TO RELATED APPLICATIONS

This Non-provisional application claims priority under 35 U.S.C. §119(a) on Patent Application No(s). 100106087 filed in Taiwan, Republic of China on Feb. 23, 2011, the entire contents of which are hereby incorporated by reference.

### BACKGROUND OF THE INVENTION

1. Field of Invention

The present invention relates to a composition, kit and use for immune enhancement. More particularly, the present invention relates to an immune enhancement composition, kit and use composed of two different materials.

2. Related Art

Rapamycin, also known as sirolimus, was isolated from Streptomyces hygroscopicus of the soil sample obtained from Easter Island. Rapamycin has demostrated anti-fungal activity both in vitro and in vivo, especially in Candida albicans, and thus is used as an agricultural antifungal agent in the early days. However, when the fact that rapamycin has an inhibition effect against immunoreaction and anti-proliferative effect has been discovered, rapamycin is used on large scale as an immuno-inhibition drug, to inhibit the rejection of, for example, acute allotransplantation.

There are a variety of commercial drugs containing rapamycin now, for example, RAPAMUNE® (Wyeth, Collegeville, Pa., USA), the main use of which is for preventing organ rejection after kidney transplant. In addition, CERTICAN® produced by Novartis, East Hanover, N.J., USA, also provides the effect of preventing organ rejection.

On the other hand, chloroquine is a substituted quinoline which has been commonly used for over 60 years for the prevention and treatment of malaria prophylaxis, or for rheumatoid arthritis treatment and HIV treatment.

However, there has been no report or article of using a combination of rapamycin and substituted quinoline to promote the activity of immune system.

### SUMMARY OF THE INVENTION

In view of the deficiency of prior art, the present invention is developed. An objective of the present invention is to provide a composition, kit and use for immune enhancement, which is substantially achieved by using the combination of rapamycin and substituted quinoline.

Another object of the present invention is to provide an immune enhancement composition, kit and use for cancer or tumor therapy, which can substantially be deemed as a composition, kit and use that can be applied to cancer immune therapy to promote the immune ability of an individual in need and thus achieve the goal of treating or preventing the formation of a cancer or a tumor.

Another object of the present invention is to provide an immune enhancement composition for the treatment or prevention of microbe or viral infections achieved by promoting the immune ability of an individual in need.

An immune enhancement composition according to the present invention includes rapamycin, substituted quinoline and a pharmaceutically acceptable carrier, diluent, excipient or a combination thereof.

An immune enhancement kit according to the present invention includes rapamycin and a first pharmaceutically acceptable carrier, diluent, excipient or the combination thereof; and substituted quinoline and a second pharmaceutically acceptable carrier, diluent, excipient or a combination thereof.

A use of a combination of a rapamycin and substituted quinoline of the present invention is as an immune enhancement composition.

In order to demonstrate the technical features of the present invention in the follow-up contents, specific terms are defined hereunder, and the detail of the present invention will be illustrated thereafter. In addition, both the immune enhancement composition and immune enhancement kit of the present invention contain rapamycin and substituted quinoline. In the invention, “a combination of rapamycin and quinoline” is considered to include the immune enhancement composition and the product of the preparation of an immune enhancement kit.

As used herein, an “immune enhancement composition” refers to a composition containing at least two materials. Preferably, it refers to a composition containing an effective amount of two materials and a pharmaceutically acceptable carrier, diluent, excipient or a combination thereof. Among which, the composition aforementioned can promote the immune ability of an individual in need to resist the development of a disease or an adjustment disorder, and then achieve the goal of eliminating, inhibiting, improving, comforting or preventing a disease or an adjustment disorder and its root or symptoms. Among which, the composition aforementioned may be a combination of rapamycin and substituted quinoline.

To continue, “to promote the immune ability of an individual in need” refers to improve, enhance, intensify, regulate or recover the ability of the immune system of an individual, especially the ability of reaction and function on the factors causing a disease or an adjustment disorder. For example, the result of the abovementioned can be exemplified but not limited to enhancing or inducing non-specific immune response, preferably, by increasing the activity of natural killer (NK) cells in an individual in need to produce cytotoxicity to the target.

[Rapamycin]

As used herein, “rapamycin” refers to one selected from a group of compounds containing basic rapamycin structure (Formula I), including the derivatives obtained from chemical/biological modification or substitution, while still maintaining the nature of the original basic rapamycin structure or possessing similar properties with that of the original basic rapamycin structure. Therefore, “rapamycin” substantially comprises easters, ethers, enzymes, hydrazones, hydroxylamines, and rapamycins with the functional groups of the basic rapamycin structure modified by reduction, oxidation or substitution. Needless to say, “rapamycin” also includes pharmaceutically acceptable salts formed from the acidic/basic bases of Formula I.

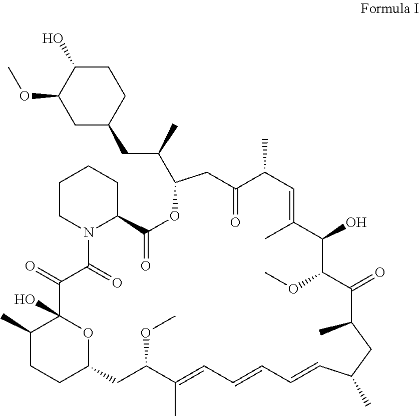

Rapamycin is referred to sirolimus, which is well-known in the present field, and it also comprises other compounds with identical or similar structures but different trade names. Further, except rapamycin, it is also proper for the present invention to use a compound selected from the group consisting of everlimus, temsirolimus, tacrolimus, 2-(dimethyl phosphinyl)sirolimus (deforolimus), biolimus and (42S)-42-deoxy-42-(1H-tetrazol-1-yl)-rapamycin (zotarolimus).

[Substituted Quinoline]

As used herein, “substituted quinoline” not only refers to chloroquine, but also includes, but not confines to, derivatives or chloroquine homologue obtained by further modification or substitution of chloroquine. Among which, derivatives obtained by further modification or substitution of chloroquine can possess the chemical structure of chloroquine, but one or more hydrogens or functional groups thereof are modified or substituted by one or more substituents. Specifically, the substituents mentioned above can be halogen, C₁₋₁₀ alkyl, —OC₁₋₁₀ alkyl, hydroxyl, C₆₋₁₀ aryl, heteroaryl, heterocycloalkyl, alkheterocycloalkyl, heteroalkyl or alkheteroalkyl. For example, substituted quinoline can be selected from a group consisting of free hydroxychloroquine, primaquine, amoproquine, amodiaquine, cycloquine, sontoquine, quinacrine, tebuquine and bis-pyroquine.

[Compound]

All of the compounds or materials, which are mentioned herein and substantially related to the technical feature of present invention, include the compounds or materials themselves and any pharmaceutically acceptable forms thereof. Among which, the pharmaceutically acceptable forms can be exemplified, but not limited to, various isomers including diastereomers and enantiomers, salts, ionization forms, solvent, prodrug, polymorph and racemic mixture.

[Ratio and Usage]

For the combination of rapamycin and substituted quinoline to perform immune enhancement effect, the weight percentage ratio of an effective amount of rapamycin and substituted quinoline in an immune enhancement composition can be between about 1:6×10⁸ and about 2000:1. However, the enhancement effect of different ratio within the scope can be greatly different, thus, the advisable ratio of application is between about 1:100 and about 1:5000, preferably is between about 1:1000 and about 1:3000, more preferably is about 1:2000. Needless to say, said ratio can also be applied to the pharmaceutical composition of the present invention, or can be used as the preparation ratio of the immune enhancement kit of the present invention when it is used in practice.

An example according to the ratio stated above is described hereinbelow. When the combination of rapamycin and substituted quinoline are prepared as a solution form for oral administration or injection, the effective amount of rapamycin may range from about 1 pg/ml to about 1 μg/ml, and the effective amount of substituted quinoline may range from about 0.5 ng/ml to about 0.6 mg/ml. Preferably, the effective amount of rapamycin may range from about 100 pg/ml to about 10 ng/ml, and the effective amount of substituted quinoline may range from about 10 ng/ml to about 15 μg/ml.

Among which, “an effective amount” refers to an amount of the materials or the compounds mentioned, which can promote or intensify the immune ability or immune response of an individual in need, preferably, can treat a disease or an adjustment disorder or prevent the formation of a disease or an adjustment disorder. As used herein, “an individual in need” refers to an animal suffering from a disease or an adjustment disorder or having a tendency of suffering from a cancer or an adjustment disorder. Said animal includes mammals and preferably refers to humans.

Therefore, as a person having ordinary skills in the art can understand, there may be several applications for an effective amount of a combination of rapamycin and substituted quinoline, for example, the treatment or prevention of a disease or an adjustment disorder, preferably the treatment or prevention of a cancer or a tumor. Of course, it also adapts to the treatment or prevention of a disease or an adjustment disorder caused by microbe or virus infections, however, the present invention is not limited to this. When considering the importance, however, the contents and embodiments stated hereunder are all examples of using the combination of rapamycin and substituted quinoline for the treatment or prevention of a cancer or a tumor, for illustration and references for other types of diseases or adjustment disorders.

In a treatment or prevention of a cancer or a tumor, the combination of rapamycin and substituted quinoline can be administrated individually or applied together with other therapy methods, for example, a radiation treatment or a chemical treatment. On the other hand, when applying together on an individual in need, the combination of rapamycin and substituted quinoline can be applied with other therapy methods at the same time or at a different time of the day, for example, with an interval of 1 or 5 hours. Similarly, during the process of combinational application, the combination abovementioned may also be applied before, during or both before and during the application of other therapy methods, however, the present invention is not limited to this.

Besides, the frequency and order of the application of the combination of rapamycin and substituted quinoline and other therapy methods are not limited. During one combinational therapy process only one administration of the combination of rapamycin and substituted quinoline is required, or the combination of the two of them is administrated every time before the application of other therapy methods, or is re-administrated during the period of every two, three or five times of administration of other therapy methods. However, the present invention is not limited to this, if only providing the better treatment or prevention effect.

To sum up, the immune enhancement composition, kit and use according to the present invention use the combination of rapamycin and substituted quinoline to promote the immune ability of an individual in need to complete the goal of treating or preventing a disease or an adjustment disorder.

### BRIEF DESCRIPTION OF THE DRAWINGS

The invention will become more fully understood from the detailed description and accompanying drawing, which are given for illustration only, and thus are not limitative of the present invention, and wherein:

FIG. 1 is the data map of activity of NK cells analyzed by a Flow cytometer.

### DETAILED DESCRIPTION OF THE INVENTION

The present invention will be apparent from the following detailed description, which proceeds with reference to the accompanying drawings, wherein the same references relate to the same elements. The ratio and usage of rapamycin, substituted quinoline and the combination thereof can be referred to the paragraphs above and thus will not be re-illustrated here.

An immune enhancement composition according to the present invention comprises rapamycin and substituted quinoline and a pharmaceutically acceptable carrier, diluent, excipient or a combination thereof. In the present embodiment, the rapamycin is a compound of Formula I described above, and the substituted quinoline is chloroquine.

The method of preparing rapamycin and substituted quinoline is well-known by a person having ordinary skill in the art. Further, from the disclosure of the present Specification, it is easy to understand the technique of producing an immune enhancement composition from both rapamycin and substituted quinoline with a pharmaceutically acceptable carrier, diluent, excipient, or a combination thereof. Among which, the pharmaceutically acceptable carrier, diluent or excipient can be, for example, well-known magnesium carbonate, magnesium stearate, talc, sugar, lactose or a combination thereof.

The preparation of an immune enhancement composition is not limited to uniformly mixing rapamycin and substituted quinoline, that is, in immune enhancement composition with the same dosage form, rapamycin and substituted quinoline can be mixed in other ratio, or even not being mixed. For example, an immune enhancement composition can be a tablet or a capsule, wherein part of it being rapamycin and another part of it being substituted quinoline. Preferably, in the present embodiment, rapamycin and chloroquine can be bought from a vendor (please refer to the experimental examples), and an immune enhancement composition can be powders prepared by mixing powdery rapamycin and substituted quinoline with a pharmaceutically acceptable diluent.

Generally speaking, one with ordinary skill in the art can easily know the compatible dose range of rapamycin and substituted quinoline, which is also the compatible dose range of the present invention. For example, the acceptable dose range of rapamycin for an individual in need is between about 0.001 mg and about 1000 mg per kg per day, and the acceptable dose range of substituted quinoline for an individual in need is between about 0.001 mg and about 1000 mg per kg per day. Preferably, the dose range of rapamycin is between about 0.1 mg and about 100 mg per kg per day, and the dose range of substituted quinoline is about 1.5 mg to about 150 mg per kg per day. Certainly, it is essential for the dose of rapamycin or substituted quinoline to alter according to the administration pathway, the physiological condition of an individual in need, or the therapy method applied together with. Generally speaking, oral administration requires higher dose. Generally, it requires relatively lower dose during the primary stage of the treatment.

There may be a dose unit of an immune enhancement composition in every formulation, that is, one formulation contains enough dose for performing sensitizing effect in an individual in need, as is convenient for direct administration. In the present embodiment, every package of powders contains a dose unit of immune enhancement composition. Needless to say, in other embodiments of the present invention, one dose unit of an immune enhancement composition can be separated into several sub-dose units or sub-packages, for example, separated into two to three tablets or capsules and packed in the same blister pack.

In the present embodiment, the immune enhancement composition is produced independently and thus can be administrated independently to an individual in need to promote its immune ability. However, in other embodiments of the present invention, an immune enhancement composition may further contain an effective amount of active composition of other types therapy methods, so that other than promoting immune ability, it also provides the effect of treating a disease or an adjustment disorder.

The following embodiment uses the combination of rapamycin and substituted quinoline for the treatment or prevention of a cancer or a tumor to explain how to apply the immune enhancement composition specifically. Of course, as stated above, a combination of rapamycin and substituted quinoline may also be used for preventing or treating a microbe or viral infection, but the specific application and details are similar to when it is applied for a cancer or a tumor and can be referred to hereunder.

In the present embodiment, an immune enhancement composition can be used in the treatment or prevention of any form of cancer or tumor, especially lung cancer, colon cancer or breast cancer, especially all kinds of solid tumor, to treat a tumor, retard the growth of a tumor or prevent the formation of a tumor. For the dosage form of an immune enhancement composition, the combination of rapamycin and substituted quinoline can be produced with a pharmaceutically acceptable carrier, diluent, excipient or a combination thereof, into, for example but not limited to, a solid or liquid oral dosage form, such as a tablet or a capsule, preferably an oral administrative tablet. As for other compatible dosage forms, there are pills, sachets, granules, powders, chewing gums, suspensions, emulsions, suppositories and solutions.

Except oral administration, in other embodiments, an immune enhancement composition can also be administrated via a pathway outside the gastrointestinal tract, for example but not limited to, intravenous injection, or subcutaneous, intramuscular, intrathecal, intraperitoneal, intrarectal, viginal, nasal, intragastric, intratracheal, pulmonary, intratumoral or peritumoral injection or implantation.

When administrating to an individual in need, the rapamycin in an immune enhancement composition can cause a synergistic effect with the substituted quinoline resulting in a even more remarkable enhancement effect comparing to when rapamycin or substituted quinoline is used individually. Specifically, a combination of rapamycin and substituted quinoline can enhance the activity of NK cells in an individual in need through the synergistic effect between them and thus promote its immune ability. An NK cell can cause non-specific cytotoxicity to a cancer cell, a variant cell, or a tumor cell, therefore, through the enhancement of its activity, it may achieve the goal of eliminating, inhibiting, improving, comforting or preventing a cancer and its symptoms; retarding, prohibiting, reversing the rate of tumor proliferation; or the medical effects similar to the foregoing goals.

According to the above, although it is prior art to use rapamycin or substituted quinoline individually as the active composition of a cancer therapy, that is, an anti-cancer or an anti-tumor medicine, but the treatment effect is very restricted. The applicant of the present invention discovered that the combination of rapamycin and substituted quinoline is not a compatible active composition that provides treatment effect in a cancer treatment procedure, but ought to play an assistant part, that is, the immune enhancement composition disclosed in the present invention. The combination of rapamycin and substituted quinoline can achieve the goal of treating or preventing the formation of a cancer or a tumor by promoting the ability of the immune system, and thus it should be deemed as a cancer immune therapy composition.

In yet another embodiment, the present invention provides an immune enhancement kit. In the present embodiment, the kit can be similarly used for the treatment or prevention of a disease or an adjustment disorder, preferably for the treatment or prevention of a cancer or a tumor, and increase the activity of NK cells in an individual in need by the combination of rapamycin and substituted quinoline to achieve the goal. Of course, an immune enhancement kit may also be applied in other aspects by its function of promoting immune ability. The present invention is not limited to this. In addition, an immune enhancement kit may similarly be applied individually or together with other therapy methods.

For the components of the composition, an immune enhancement kit includes a rapamycin and a first pharmaceutically acceptable carrier, diluent or excipient; and a substituted quinoline and a second pharmaceutically acceptable carrier, diluent or excipient. Among which, the related description of the rapamycin, the substituted quinoline and the rest can be referred to in the paragraphs above and thus will not be re-illustrated here. Only the insufficient or unexplained parts will be further illustrated.

The first and the second pharmaceutically acceptable carrier, diluent, excipient or a combination thereof can be a well-known material or composition in the art, and the method and dosage form of preparing a prescription of each of them with rapamycin or substituted quinoline are also prior art in the field of the present invention. In particular, in the present embodiment, there can be an individual pack or container, such as a blister pack, for an immune enhancement kit, to contain or store, for example, the tablet made of rapamycin and a first pharmaceutically acceptable excipient and the tablet made of substituted quinoline and a second pharmaceutically acceptable excipient. Said individual pack can be provided to an individual in need during application or be administrated to an individual in need after preparation.

Needless to say, in other embodiments, the rapamycin and the substituted quinoline can be processed together into an injection form, which is obatined by preparing according to a specific ratio at the time of administration or within a suitable time period before the administration, for example, within one to several weeks (such as within 10 days, 5 days or 24 hours). Accordingly, when rapamycin and substituted quinoline need to be administrated separately, in different dosage forms, or when the compositional ratio of which needs to be adjusted, it is especially beneficial to use the immune enhancement kit of the present invention.

In yet another embodiment, the present invention provides a use of a combination of rapamycin and quinoline as a sensitizer, wherein the related description of the rapamycin, the substituted quinoline and other parts can be referred to in the paragraphs above and thus will not be re-illustrated here.

As mentioned above, according to the immune enhancement composition, kit and use of the present invention, a combination of rapamycin and substituted quinoline is used to increase the immune ability of an individual in need and then achieve the goal of treating or preventing a disease or an adjustment disease.

### EXPERIMENTAL EXAMPLE 1

### To Prepare the Immune Enhancement Composition

The rapamycin and chloroquine used in the present invention were purchased individually from Sigma-Aldrich, Inc, St. Louis, Mo., USA. Obtain about 1 mg of rapamycin and about 2 g of substituted chloroquine by a weighting scale in room temperature, mix the powder rapamycin and chloroquine uniformly in a weight percentage ratio of 1:2000. And then, pack it into a dosage form of powders or sachets and store in room temperature.

### EXPERIMENTAL EXAMPLE 2

### Using the Immune Enhancement Composition to Enhance the Activity of NK Cells

Select Human erythromyeloblastoid leukemia cell line K562 as target cells and culture it by tissue culture technique in RPMI 1640 supplemented with 10% FBS to a proper amount. And then, take a suspension containing 1×10⁶ cells and inoculate it in a 6-wells plate. In addition, prepare 10 nM rapamycin, 10 μM chloroquine separately in room temperature and similarly add the two of them together in room temperature in sequence. And then, put the plate into a 37° C. incubator to continue culturing for 48 hours.

Use 1 mM of green fluorescent cell linker dye PKH67-GL(Sigma-Aldrich), which resolves in alcohol, to label the aforementioned cells. The detail steps are: resuspend 1×10⁶ cells in 1 ml of Diluent C(Sigma-Aldrich), add PKH67-GL until the density reaches 4×10⁻⁶ M and dye for 4 minutes under 25□. Add 1 ml FBS and 2 ml complete medium to end the coloring reaction, then wash it with PBS for three times. And then, dilute the amount of cells in a complete medium until it reaches 2.5×10⁵ cells per ml and confirm the labeling and activity of cells by a Flow cytometer.

Dilute the amount of NK cells (the effector cell of the present Experimental Example) to 6.25×10⁶ per ml. Mix 100 μl of the aforementioned diluted cell solution with 100 μl of the target cell solution so that the ratio of effector cells and target cells reaches 25:1. Put the mixed cells in a cell culture and culture for 16 hours in a 37□ incubator with 5% CO₂. Use 2 μl of 0.5 mg/ml propidium iodide (PI) (Sigma-Aldrich) dissolved in PBS to re-suspend the cells 5 minutes before the end of the culture cycle and complete the coloring of dead cells. Analyze the activity of NK cells by a Flow cytometer twice for each group, 10⁴ cells each time.

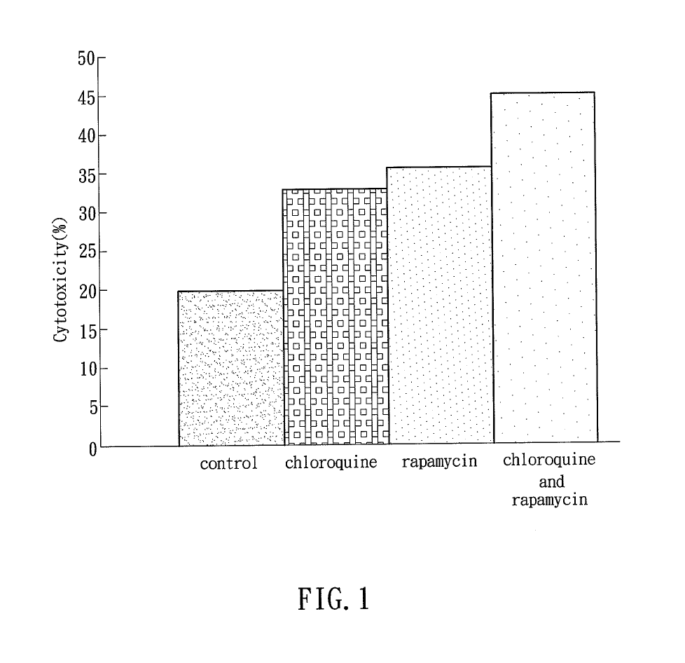

FIG. 1 is the data map of activity of NK cells analyzed by a Flow cytometer. Referring to FIG. 1, the one marked as “Control” is the activity ratio of NK cells according to the same treatment procedure but with no addition of chloroquine or rapamycin to the target cells; the ones marked as “chloroquine” or “rapamycin” are individually the activity ratio of NK cells according to the same treatment procedure with the either chloroquine or rapamycin, and the one marked as “chloroquine and rapamycin” means the activity ratio of NK cells under the treatment of both compounds. It is clear from the data that when the target cells are under the treatment of both compounds at the same time, the activity ratio of NK cells is 25% higher than Control, however, when under individually treatment of either compound, the activity ratio of NK cells may only increase by 12.5% or 15%.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An immune enhancement composition, comprising rapamycin, substituted quinoline, and a pharmaceutically acceptable carrier, diluent, excipient or a combination thereof.

2. The immune enhancement composition of claim 1, wherein the rapamycin is a compound of the following formula:

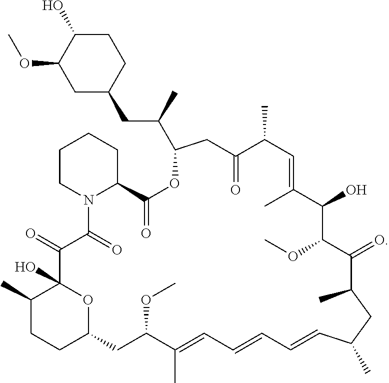

3. The immune enhancement composition of claim 1, wherein the substituted quinoline is a chloroquine.

4. The immune enhancement composition of claim 1, wherein the weight percentage ratio of the rapamycin to the substituted quinoline is between 1:100 and 1:5000.

5. The immune enhancement composition of claim 1, wherein the rapamycin and the substituted quinoline cause a synergistic effect.

6. The immune enhancement composition of claim 1, wherein the composition is used for the treatment or prevention of a cancer or a tumor or for the treatment or prevention of microbe or virus infection.

7. The immune enhancement composition of claim 1, which increases the activity of NK cells in an individual in need.

8. An immune enhancement kit comprising:
rapamycin and a first pharmaceutically acceptable carrier, diluent, excipient, or a combination thereof; and
substituted quinoline and a second pharmaceutically acceptable carrier, diluent, excipient, or a combination thereof.

9. The immune enhancement kit of claim 8, wherein the rapamycin is a compound of the following formula:

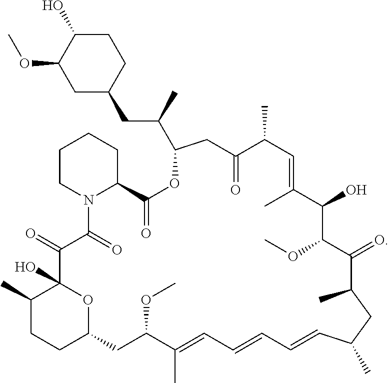

10. The immune enhancement kit of claim 8, wherein the substituted quinoline is a chloroquine.

11. The immune enhancement kit of claim 8, wherein the rapamycin and substituted quinoline cause a synergistic effect.

12. The immune enhancement kit of claim 8, wherein the immune enhancement kit is used for the treatment or prevention of a cancer or a tumor or for the treatment or prevention of microbe or virus infection.

13. The immune enhancement kit of claim 8, which increases the activity of the NK cells in an individual in need by the combination of rapamycin and substituted quinoline.

14. A use of a combination of rapamycin and substituted quinoline as an immune enhancement composition.

15. The use of claim 14, wherein the rapamycin is a compound of the following formula:

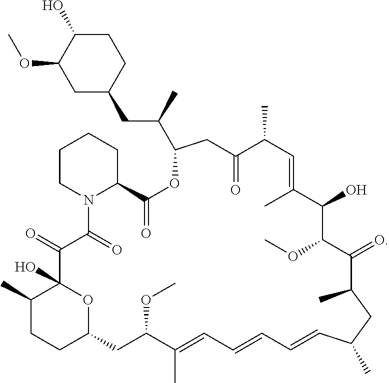

16. The use of claim 14, wherein the substituted quinoline is a chloroquine.

17. The use of claim 14, wherein the weight percentage ratio of the rapamycin and the substituted quinoline is between 1:100 and 1:5000.

18. The use of claim 14, wherein the rapamycin and the substituted quinoline cause a synergistic effect.

19. The use of claim 14, wherein the immune enhancement composition is used for the treatment or prevention of a cancer or a tumor or for the treatment or prevention of microbe or virus infection.

20. The use of claim 14, wherein the immune enhancement composition increases the activity of NK cells in an individual in need by the combination of rapamycin and substituted quinoline.

21. The use of claim 14, wherein the immune enhancement composition is used for treating a solid tumor.

22. The use of claim 14, wherein the immune enhancement composition is a solid oral administration or a liquid oral administration.
